(19) United States
(12) Patent Application Publication
(10) Pub. No.: US 2005/0165390 A1
(43) Pub. Date: Jul. 28, 2005

(54) Apparatus for electrosurgery

(76) Inventors: Aldo Mauti, Aprilia LT (IT); Paolo Mauti, Aprilia LT (IT)

Correspondence Address: Leffert Jay & Polglaze, P O Box 581009, Minneapolis, MN, 55458-1009, US

(21) Appl. No.: 10/504,212

(22) PCT Filed: Feb. 11, 2003
(86) PCT No.: PCT/IT03/00070

(30) Foreign Application Priority Data
Feb. 11, 2002 (EP) 02425066.4

(51) Int. Cl. A61B 18/14

(52) U.S. Cl. 606/41

(57) ABSTRACT

Apparatus for electrosurgery (1) of a type apt to the treatment of neoplastic tissues by thermoablation, comprising at least two pairs of electrodes (4,5,6,7;41,51,61,71) and a generator of electrosurgical current (2,3,) to make a thermoablation current flow between the electrodes according to a bipolar thermoablation mode.

### DESCRIPTION

The present invention relates to an apparatus and a device for electrosurgery, and in particular to an apparatus and to a device for the treatment of neoplastic tissues by thermoablation.

The invention further refers to a corresponding method for the treatment of neoplastic tissues by thermoablation.

One of the therapies for the cure of primary or metastatic tumours, above all at a hepatic and pulmonary level, consists in the electrosurgical ablation of the tumour tissue targeted by the treatment, a tissue typically consisting of a nodule.

The electrosurgical apparatuses for generating said thermoablation effect mainly consist of a generator of alternating and high-frequency electrosurgical current, delivered by means of a so-called active electrode. The latter is generally needle-shaped and placed percutaneously or with an open approach onto the target tumour tissue to be eliminated. The thermoablation is attained as, near to the active electrode an elevated current density and therefore a substantial increase in temperature by Joule effect is generated, which in turn devitalizes the cells in a region surrounding the electrode itself.

As it is well-known to a person skilled in the art, the electrosurgical apparatuses at issue provide, among the other things, a so-called monopolar thermoablation according to which the circuit of the electrosurgical current delivered by the active electrode is made via a so-called neutral or return electrode which has a very wide contact area with the patient's skin and is placed at a region of the patient's body totally separated from the region concerned by the actual surgery.

The present operation modes in the tumour tissue thermoablation surgery entail several drawbacks.

One of the main drawbacks lies in that said modes merely enable the treatment of a very limited tissue portion at a time. In particular, the region concerned by the thermoablation is reduced to a small neighbourhood of the point of delivery of the current by the active electrode. This is also due to the fact that the dehydration which onsets near the active electrode impedes the continuation of flow of the thermoablation current. In fact, in the known systems, near to the site of current delivery by the active electrode, there ensues an elevated temperature gradient, with a consequent carbonization of the tissues directly contacted by the active electrode and with a marked dehydration of the tissues surrounding the electrode itself, which impedes the current to continue to flow.

In order to prevent this excessive drying of the tissues in the vicinity of the active electrode various contrivances have been devised, like, e.g., a slow heating of the active electrode apt to promote heat propagation, a forced water-cooling of the electrode itself, or a cooling of the tissue by a flow of physiological water.

However, even with these contrivances the diameter of the treated tumour cannot be greater than about 2-3 cm.

The technical problem underlying the present invention is to provide an apparatus and a device for electrosurgery enabling to overcome the drawbacks hereto mentioned with reference to the known art.

This problem is solved by an apparatus for electrosurgery according to claim 16.

According to the same inventive concept, the present invention further relates to a device for electrosurgery according to claim 1.

Always according to the same inventive concept, the invention further provides a method for the treatment of neoplastic tissues by thermoablation according to claim 29.

In the present context for ‘constructive interference’ of the thermoablation currents it is meant that the related thermoablation heats add up.

The present invention provides several relevant advantages. Firstly, the attainment of a constructive interference between the thermoablation currents delivered by the electrode pairs of the apparatus or of the device of the invention—i.e. the fact that the related thermoablation effects add up—enables a reduction of the current intensity that each electrode has to deliver in order to attain the desired thermoablation. Thus, the dehydration of the tissues surrounding each electrode is reduced, and this of course promotes the flowing of the electrosurgical current in a region wider than that allowed by the abovedescribed known systems.

In the specific quadrilateral arrangement of the electrodes of the device of the invention, in which the electrodes of each pair be arranged at opposed vertexes of the quadrilateral, the currents interfere constructively at the central portion of the quadrilateral area, i.e. just whereat the maximum thermoablation effect is required. Another significant advantage lies in that the provision of a plurality of electrode pairs powered according to a bipolar thermoablation mode enables to widen the viable dimensions for the target region. Moreover, said electrode pairs and the bipolar powering mode thereof enable a great versatility in the extension and shape of the treatable tumour regions, in the selection of the route for the thermoablation currents and in the electrode placement.

Other advantages, features and the modes of employ of the present invention will be made apparent in the following detailed description of some embodiments thereof, given as a non-limiting example. Reference will be made to the figures of the attached drawings, wherein:

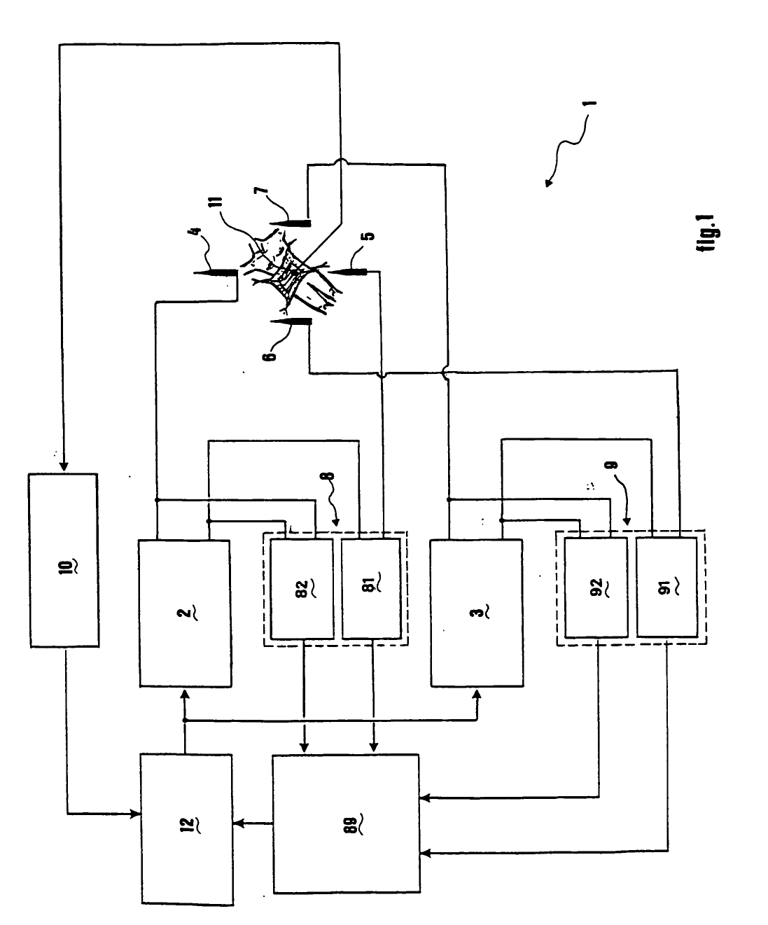

FIG. 1 is a block diagram of a first embodiment of the apparatus for electrosurgery according to the present invention;

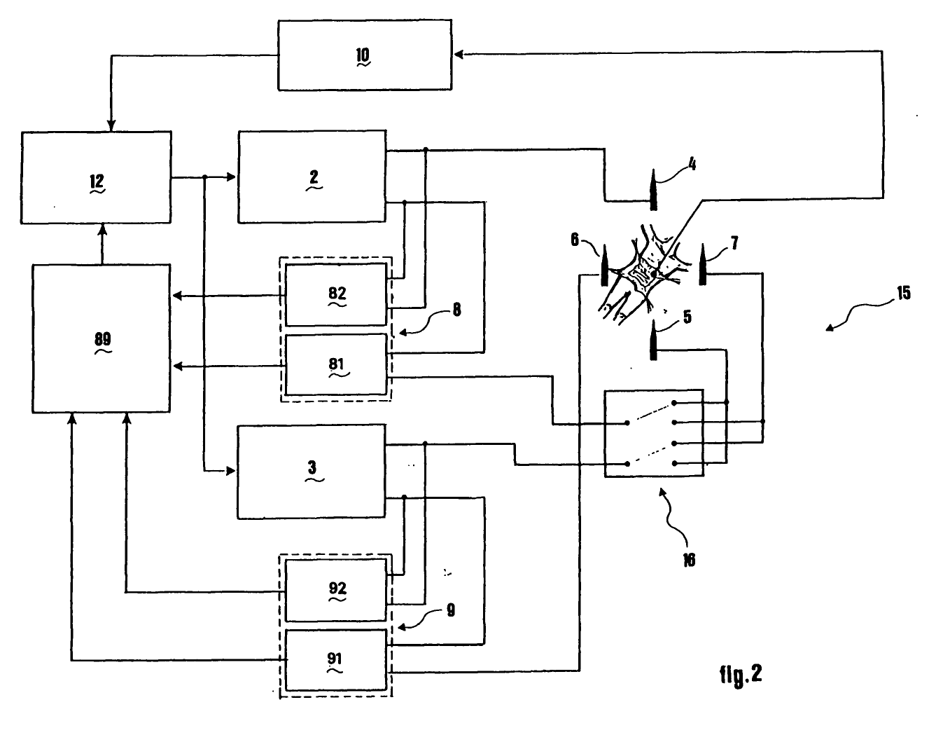

FIG. 2 is a block diagram of a second embodiment of the apparatus for electrosurgery according to the present invention;

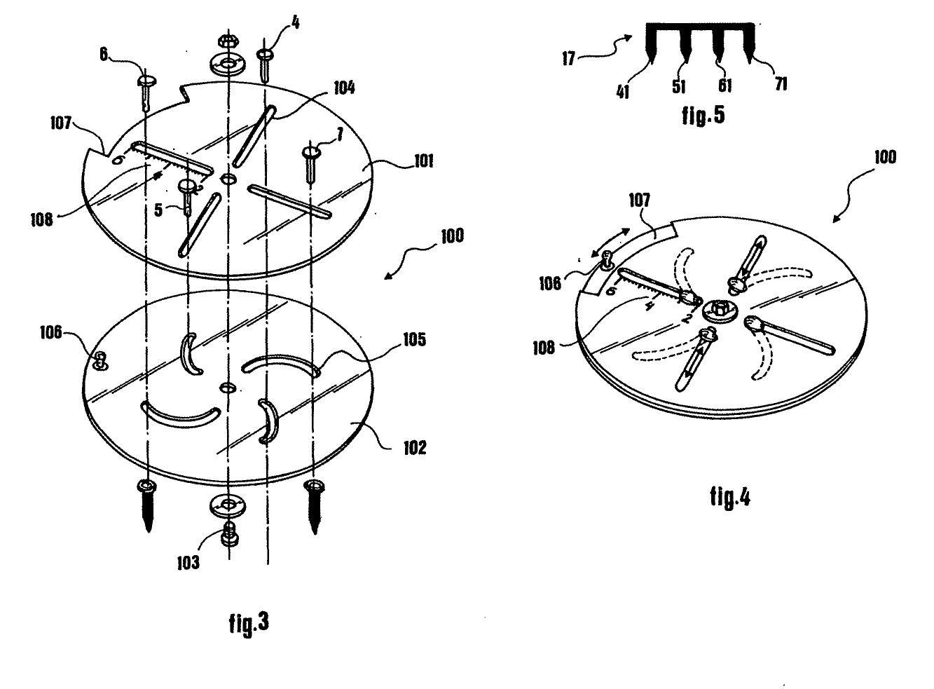

FIG. 3 is an exploded perspective view of a device for electrosurgery according to the present invention;

FIG. 4 is a perspective view of the device of FIG. 3 in an assembled configuration; and

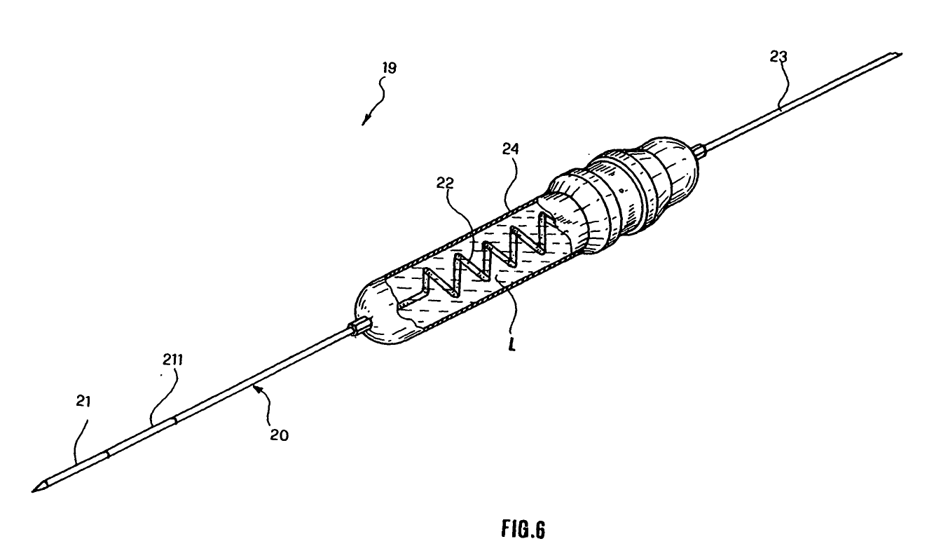

FIG. 5 is a perspective view of a comb electrode, useful when combined with the apparatus for electrosurgery according to the present invention;

FIG. 6 is a side view of another type of electrode useful when combined with the apparatus for electrosurgery according to the present invention; and

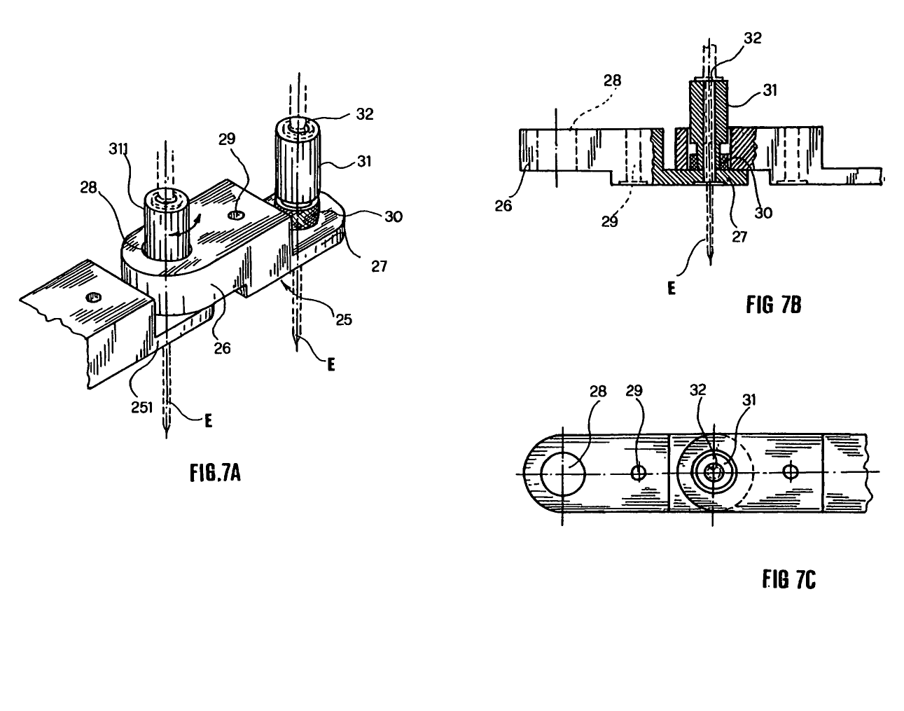

FIGS. 7A, 7B and 7C relate to an electrode supporting system useful when combined with the apparatus for electrosurgery according to the present invention, showing a perspective view, a partially sectional side view and a plan view of a component thereof, respectively.

With initial reference to FIG. 1, a first embodiment of the apparatus for electrosurgery according to the invention is generally indicated by 1. The apparatus 1 comprises means for generating an electrosurgical current, incorporated in an outside casing of the apparatus itself (the latter not shown in the figures). In particular, in the present embodiment such means are made by a first and a second current generator, indicated by 2 and 3, respectively, electrically insulated the one from the other and apt to generate currents having substantially different frequencies. Due to reasons that will be made apparent hereinafter, these frequencies preferably differ of at least 100 kHz.

By way of example, the maximum power deliverable by each generator 2, 3 can be of about 75W, on an impedance of about 200 Ohm seen therefrom.

Each of such generators 2 and 3 feeds a respective pair of electrodes according to a bipolar operation mode of each of such pairs. In particular, the apparatus 1 comprises a first and a second electrode, indicated by 4 and 5, powered by the first generator 2 and a third and fourth electrode, indicated with 6 and 7, respectively, and powered by the second generator 3.

These electrodes 4, 5, 6 and 7 can be permanently connected to the respective generator 2 or 3 or be removably connectable thereto by suitable ports provided at the outer casing of the apparatus 1. In the latter case, the casing containing the two generators 2 and 3 and the further components of the apparatus 1 which will be described could be provided to an end user separately from said four electrodes 4, 5, 6 and 7.

Preferably, each of the electrodes 4, 5, 6 and 7 has a coating of anti-stick material or it has been subjected to an anti-stick treatment at the related current delivering portion, in order to avoid the adhering of the treated tissue thereto.

Moreover, always according to a preferred embodiment, the electrodes 4, 5, 6 and 7 are made of brass and have the tip end electrically insulated or made of an insulating material. The insulating tip is aimed at preventing the reduction of the electrode size in the zone thereof from producing a current density such as to char the treated tissue. Brass adoption avoids the need for cooling of the electrodes themselves or of the treated body region.

Alternative embodiments can provide electrodes made of any metal sufficiently rigid to enable penetration into the body tissue without bending and capable of conducting heat so as to remove that generated during the treatment near the electrode.

As it will further be illustrated later on, the overall configuration of the generators 2 and 3 and of the related electrode pairs 4, 5 and 6, 7 is such that, during its surgical employ, there be generated a constructive interference between the electrosurgical currents delivered by the two pairs of electrodes, i.e. that the respective thermoablation effects add up.

The apparatus 1 further comprises first and second impedance determining means, respectively combined with the first and to the second generator 2 and 3 and indicated by 8 and 9, respectively. Said means 8 and 9, analogous to the corresponding means already present in the electrosurgical apparatuses of the known art, provide each a current measuring unit, 81 and 91 respectively, apt to measure the current flowing in the circuit made by the respective pair of electrodes 4 and 5 or 6 and 7 and by the respective generator 2 or 3. This impedance determining means 8 and 9 further provide a voltage measuring unit, 82 and 92 respectively, apt to measure the voltage associated with said circuit across the respective generator 2 or 3.

The apparatus 1 further comprises an impedance computing unit 89 common to the first and second impedance determining means 8 and 9. This latter unit 89 is inputted the data related to the voltage and current measurements carried out by the voltage measuring units 82 and 92 and by the current measuring unit 81 and 91, and it outputs the corresponding impedance values.

As it is known, this impedance determining is carried out both to determine the effect of the thermoablation treatment and for safety reasons, i.e. to control the regular current flow in the region concerned by the thermoablation.

It will be appreciated that the determining of the impedance between each pair of electrodes 4, 5 and 6, 7 enables to monitor the treatment uniformity.

The apparatus 1 further comprises a temperature measuring unit 10, which in turn provides a temperature sensor 11 apt to be applied at a central portion of the region concerned by the thermoablation in order to measure the local temperature thereof.

As in the case of the electrodes 4, 5, 6 and 7, also the sensor 11 can be removably connectable to further components of the related unit 10 internal to the outside casing of the apparatus 1.

The apparatus 1 further comprises a control unit 12 for controlling the delivered power, connected to the temperature measuring unit 10 and to the impedance computing unit 89, apt to control the power delivered by the generators 2 and 3 as a function of the data outputted by said units 10 and 89. In particular, the control unit 12 is apt to modulate the strength of the current outputted by the generators 2 and 3 as a function of the actual temperature and impedance values, in order to prevent as much as possible damage to healthy tissues.

Since the impedance determining means 8 and 9, the temperature measuring unit 10 and the control unit 12 are made with hardware and/or software components and according to modes well-known to a person skilled in the art, a further description thereof will be omitted.

Hereinafter, the operation modes of the hereto described apparatus 1 will be illustrated.

As it is schematically shown in FIG. 1, preferably the electrodes 4, 5, 6 and 7 are located onto the neoplastic tissue to be eliminated at the vertexes of a quadrilateral, and in particular of a square, in this latter square being equidistant. In this arrangement the electrodes of each pair 4, 5 or 6, 7 are placed at opposed vertexes of said quadrilateral.

Then, the two generators 2 and 3 are driven by the control unit 12 so as to make a respective alternated high-frequency thermoablation current flow between each pair of electrodes 4, 5 and 6, 7, said currents being apt to constructively interfere at the central region of the quadrilateral area.

The abovedescribed quadrilateral arrangement and the bipolar thermoablation mode attained with each pair of electrodes 4, 5 and 6, 7 make the related thermoablation currents overlap at the central zone of the quadrilateral itself, i.e. at the central zone of the tumour tissue to be eliminated. In particular, as abovementioned, the overall configuration and the electrosurgical current delivery modes are such that the intensity of the current delivered between each pair of electrodes adds to the intensity of the current delivered by the other pair of electrodes at said central zone of the quadrilateral, i.e. such that a constructive interference between such currents so as to add up the thermoablation heats thereof is generated.

Apparently, this constructive interference between thermoablation currents can also be attained with an electrode arrangement according to the vertexes of a rhombus or of a rectangle, or even of an irregular quadrilateral in general in lieu of a square. However, in this latter case the thermoablation is advantageously more regular and homogeneous.

As abovementioned, said constructive interference of currents entails several relevant advantages. In particular, by virtue of said constructive interference of currents, the intensity of the current flowing between each pair of electrodes 4, 5 and 6, 7 can be reduced with respect to the known systems. In fact, since heat generation by Joule effect is proportional to the square of current intensity, an enhanced thermoablation effect is attained.

Moreover, by virtue of said reduction in strength of the current flowing between each pair of electrodes, with respect to the known systems also the overheating of the tissues surrounding the current delivery zone is reduced consequently.

All this entails a lesser dehydration of the tissues near the electrodes and an improved loss of the heat produced, bringing the diameter of the concomitantly treatable tumour region to about 5 cm.

All of these advantages are also attained by virtue of the fact that the coagulation and the dehydration begin at the central section of the treated region rather than near to the electrodes.

In short, the provision of a quadrilateral arrangement of the electrodes and of two generators insulated therebetween, each associated to a pair of opposed electrodes, enables to attain an interferential current which produces the maximum Joule effect not near to the electrodes but in the central zone of the target region defined thereby, and precisely at the intersection of the lines connecting the opposed electrodes powered in a bipolar mode. In this way, the extension of the ablative treatment is not impeded and the entire territory comprised within the quadrilateral defined by the electrodes can progressively be treated.

Furthermore, the distribution of the thermoablation current in the region to be treated is such that, in case an excessive drying of a specific zone occurs, the bipolar current can flow in a neighbouring zone.

Moreover, of course to the reduction of the current strength delivered by each electrode and required for the thermoablation there is associated also a lowering of the required voltage thereof. This implies the further advantage, with respect to the known systems, of substantially limiting the phenomena of electromagnetic interference of the apparatus for electrosurgery 1 with monitoring apparatuses like ultrasound or radioscopy ones.

The fact that the two generators 2 and 3 be insulated the one from the other and have frequencies substantially different therebetween prevents the interference between the respective currents from triggering unwanted stimulations. In fact, frequencies not strictly equal thereamong could result in low frequency beats capable of producing a patient's neuromuscular or muscular stimulation. On the contrary, in case said frequencies differ thereamong of at least 100 kHz, the beating frequency will not be able to produce a stimulation, as observed in the known d'Ansorval's studies, but a mere Joule effect.

Moreover, another advantage lies in that the impedance computed by the impedance determining means 8 and 9 is less affected by phenomena of excessive drying about the electrodes, and therefore determinable with greater precision with respect to the known systems.

Further, the area external to that delimited by the electrodes can be considered as damage-free, as only marginally affected by the current, whereas the region delimited by the electrodes can be fully and gradually treated.

Returning now to the operation modes of the apparatus 1, the quadrilateral arrangement of the electrodes 4, 5 and 6, 7 enables, among other things, to position the temperature sensor 11 centrally to the treated tumour region, as it is schematically shown in FIG. 1.

The control unit 12 can provide two distinct working modes, and precisely an operative mode, in which the thermoablation of neoplastic tissues is actually carried out, and a monitoring mode, in which weaker currents are made flow between the electrode pairs 4, 5 and 6, 7, in order to assess the actual value of the working impedance by means of said impedance determining means 8 and 9.

Of course, the bipolar mode delivery of current prevents the known problem of the possible burning of a patient's skin at the plate that typically implements the neutral electrode in a monopolar mode.

Furthermore, it will be appreciated that with the hereto described apparatus 1 different types of tumour treatment are viable, like e.g. a treatment based on the devitalization of the actual neoplastic mass, and/or a treatment based on the devitalization of a layer of normal tissue surrounding the neoplastic mass, so as to have the neoplastic cells die by starving, or a treatment preliminary to hepatic resection in order to reduce or eliminate bleeding during the latter.

In particular, since at a 5 kHz frequency the impedance of the neoplastic tissue is about thrice that of the normal tissue, whereas at 1 MHz the ratio approaches 1:1, should it be desired to directly treat the neoplastic mass, the working frequencies of the two generators could be e.g., of 950 kHz and 1050 kHz, whereas should it be desired to ‘line’ the tumour devitalizing the surrounding healthy tissue, the frequencies at issue could be, e.g., of 350 kHz and 450 kHz, thereby restricting the current diffusion preferentially to the healthy tissue.

With reference now to FIG. 2, an apparatus for electrosurgery of a second embodiment of the invention is generally indicated by 15.

Hereinafter, the apparatus 15 will be described merely with reference to the aspects differentiating it from the previous embodiment. Hence, components alike the abovedescribed ones are indicated by the same reference number. In particular, the apparatus 15 comprises a first and a second electrode 2 and 3 and two electrode pairs 4,5 and 6,7 in all analogous to the abovedescribed ones.

In a first working mode, the first and the second electrode 4 and 5 are connected to the first generator 2, whereas the third and the fourth electrode 6 and 7 forming the other pair are connected to the second generator 3.

The apparatus 15 further comprises switching means 16 apt to change the generator 2, 3 to which the second and the fourth electrodes, 5 and 7, respectively, are connected. This switching enables a thermoablation current to flow not only between opposed electrodes, but also between electrodes placed at adjacent vertexes of the quadrilateral, thereby enabling to treat also the zone interposed between said adjacent electrodes.

However, the apparatus according to the invention hereto described with reference to two main embodiments is susceptible of several further variant embodiments, some of which are indicated hereinafter.

According to a first variant, the apparatus of the second embodiment provides a greater switching versatility, e.g. involving also the first and the third electrode 4 and 6.

A second variant provides instead the presence of a single generator rather than two, apt to power both electrode pairs according to the abovedescribed bipolar operation mode and always so as to determine a constructive interference between the currents flowing between the electrode pairs.

Moreover, a third variant provides a different impedance determination mode with respect to that provided in the electrosurgical systems known to the art. In particular, it is provided that each electrode of at least one of said pairs be made of two portions electrically insulatable the one from the other. In a first operative mode, both electrode portions concur to deliver the thermoablation current. Further, the abovedescribed control unit provides a second operative mode, definable as impedance determination mode, in which the two portions of each electrode are electrically insulated. In particular, in this latter mode a first portion of each electrode of the pair is comprised in a current measuring circuit, which also comprises the respective generator, whereas a second portion of each electrode is used to meter the voltage across the two portions, totally independently from said current metering, by a high input-impedance system.

A person skilled in the art will understand that this impedance determination technique is borrowed from the body impedance measurement techniques.

Moreover, another embodiment provides that the apparatus of the invention employ more than two electrode pairs, and preferably three pairs of electrodes whose electrodes be placed at the vertexes of an hexagon. In this embodiment, preferably to each electrode pair a respective generator is associated, according to the same criteria and with the same aims already illustrated above with regard to the selection of two generators of the first two embodiments.

Lastly, a further variant provides that the apparatus of the invention may also operate in monopolar mode, therefore being provided with means for switching from the bipolar to the monopolar operative mode, as well as of a suitable connection port for a plate-type neutral electrode.

With reference to FIGS. 3 and 4, hereinafter a device for electrosurgery, generally indicated by 100, specifically apt to be employed associated with the apparatus for electrosurgery according to the invention, and in particular with one of the abovedisclosed embodiments of this apparatus will be described.

The device 100 comprises one supporting body made of two parts, and in particular formed by a first and a second body, 101 and 102 respectively, substantially flat and having the same disc-like shape. These bodies 101 and 102 are overlapped and movable, in particular rotatable, the one with respect to the other. To this end, they are connected by a central pin 103.

Preferably, the bodies 101 and 102 are made of Plexiglas.

The device 1 also comprises four electrodes, likewise indicated by 4, 5, 6 and 7, in all analogous to the abovedescribed ones. These electrodes 4, 5, 6 and 7 are constrained to the two supporting bodies 101 and 102 and are apt to be connected in pairs to means for generating an electrosurgical current, it too analogous to the abovedescribed ones.

In particular, the electrodes 4, 5, 6 and 7 are placed at the vertexes of a quadrilateral, and in particular of a square, as seen above with reference to the arrangement of the electrodes for the apparatus for electrosurgery according to the invention.

Said electrodes 4, 5, 6 and 7 cross the bodies 101 and 102 through, and are held thereby by a shape coupling. The latter provides, for each electrode 4, 5, 6 or 7, a pair of grooves 104 and 105, obtained onto the first and the second body 101 and 102, respectively, and having a substantially rectilinear and a substantially arcuate development, respectively. The arrangement of the grooves 104 and 105 of each pair is such that those are overlapped for an extension such as to allow reception of the respective electrode therethrough.

Said grooves 104 and 105 implement means for adjusting the relative distance between the electrodes 4, 5, 6 and 7. In fact, by moving the bodies 101 and 102 the one with respect to the other, the electrodes 4, 5, 6 and 7 are guided to move in a nearing/moving away direction, said direction depending on the verse of the relative motion between said bodies.

The particular shape and placement of the grooves 104 and 105 are such that the relative interelectrode distances always remain in a predetermined ratio, and in particular that the electrodes remain equidistant the one from the other.

The device 100 also comprises stopping means for limiting the excursion of the relative rotation between the bodies 101 and 102. These means consist of a engaging member 106 fixed to the second body 102, apt to abut onto the edges of a notch 107 obtained at a peripheral edge portion of the first body 101.

Lastly, the device 100 provides at least one graduation 108 apt to indicate to a user the relative distance between said electrodes.

It will be understood that variant embodiments can provide a different implementation of said means for adjusting the relative interelectrode distance.

With reference now to FIG. 5, the apparatus according to the invention can also be employed combined with a device 17 for electrosurgery of the so-called ‘comb’ type and already known to a person skilled in the art. This latter device 17 comprises a plurality of electrodes, each corresponding to a respective comb tooth. In the present embodiment, it is provided the employ of a device having four electrodes, indicated with 41, 51, 61 and 71, respectively.

These four electrodes 41, 51, 61 and 71 are connected to the current generating means of the apparatus for electrosurgery according to the invention so as to form two electrode pairs, each apt to operate according to a bipolar thermoablation mode. In particular, according to a first embodiment variant, the device 17 is connected to an apparatus comprising a single generator of electrosurgical current. In that case, each electrode pair is connected to such single generator for the power-feeding in said bipolar mode.

According to a second variant, when the apparatus comprises two current generators, as in the case of the apparatus of the abovedescribed preferred embodiments of the invention, each of said electrode pairs will be connected to a respective generator. According to the invention, the electrode pairs of the comb-shaped device 17 may be formed so as to generate said constructive interference among the related currents, i.e. a first pair by the electrodes denoted by 41 and 71 in FIG. 5 and a second pair from the electrodes denoted by 51 and 61 always in FIG. 5.

In an alternative configuration, the electrode pairs may both be made of contiguous electrodes, e.g. a first pair of electrodes denoted by 41 and 51 in FIG. 5 and a second pair of electrodes denoted by 61 and 71, always in FIG. 5.

Hereinafter, a further type of electrode employable in association with the device as well as with the apparatus of the invention will be described with reference to FIG. 6. As it is shown in this latter figure, an electrode provided with improved heat dispersion properties is generally indicated by 19. The electrode 19 comprises an elongate main body, overall indicated by 20, made of metal, e.g., of brass, gold or of a combination thereof.

The main body 20 is in turn made of a distal tip portion 21, whereat the electrode delivers current towards or onto the tissue to be treated, an intermediate portion 22 and a terminal portion 23 for the connection to further components of the apparatus or of the device of the invention.

Preferably, the tip portion 21 provides, at the actual tip of the electrode, a gold coating. Moreover, adjacently to the gold-coated portion, this tip portion preferably comprises a stem portion 211 having a coating of electrically insulating material, in order to avoid carbonization of tissues accidentally contacting this stem portion 211 during surgery.

The intermediate portion 22 has a convoluted development, in particular developing substantially helicoidally, in order to increase the heat dispersion surface.

Moreover, preferably, at such intermediate portion 22 the electrode is encased in a capsule 24 containing a refrigerating or refrigerated liquid L or another refrigeration substance. In the present embodiment, this liquid L is water that, prior to surgery, has been cooled to a predetermined temperature, so as to be capable of effectively exchanging heat with the electrode intermediate portion 22 encased in the capsule 24 during the surgery itself.

In FIGS. 7A to 7C there is represented a part of a modular system for supporting one or more electrodes, said system being apt to be employed in association with the device and/or the apparatus of the invention.

The system at issue comprises a plurality of identical module-element s, one thereof being indicated by 25 in FIGS. 7A-7C. The module-element 25 has a first and a second portion, 26 and 27 respectively, substantially plane and arranged staggered the one with respect to the other and optionally made integral the one to the other.

Onto the first portion 26 there are obtained a first and a second seat 28 and 29, respectively, each apt to receive one electrode placed substantially orthogonal to the first portion 26.

Onto the second portion 27 there is obtained a third seat 30, receiving a tubular element 31 in turn provided with an electrode seat 32 corresponding to the inner longitudinal room thereof.

The tubular element 31 serves as rotatable connection element between two contiguous module-elements of said supporting system. In particular, as it is shown in FIG. 7A, the tubular element 311 of another module-element 251 is apt to engage the first seat 28 of the module-element 25, so as to implement a pin-sleeve coupling between the two elements 25 and 251. Thus, a bike chain—like articulated supporting system is implemented. Onto this system, the electrodes, very schematically depicted in FIG. 7A and denoted by E, may be inserted in any one of the seats 28, 29 and 32 of each module-element.

Lastly, in the light of the above, it will be understood that the invention implements a novel method of thermoablation of neoplastic tissues based on the following steps:
providing at least two pairs of electrodes according to what has been disclosed hereto;
providing current generating means for generating an electrosurgical current of the hereto illustrated type; and
making an electrosurgical current flow between each pair of electrodes according to a bipolar thermoablation mode, so as to generate a constructive interference between the currents delivered by the pairs of electrodes at the tumour tissue.

Preferably, the thermoablation currents for the electrode pairs have a substantially different frequency, in the light of the aboveillustrated with reference to the first embodiment of the apparatus of the invention.

Always as aboveillustrated, the invention enables to carry out different tumour treatment types, like e.g. a treatment based on the devitalization of the actual tumour mass and/or a treatment based on the devitalization of a layer of healthy tissue surrounding the latter, so as to starve the tumour cells to death, or a treatment preliminary to hepatic resection, in order to reduce or eliminate the bleeding during the resection itself.

According to a preferred implementation of the method at issue, the electrodes of said pairs are placed onto a patient's body at the vertexes of a quadrilateral, or, in the case of three electrode pairs, of an hexagon, so as to have the thermoablation currents flow between opposed and/or adjacent electrodes, as hereto illustrated.

The present invention has hereto been described with reference to preferred embodiments thereof It is understood that there could be other embodiments referable to the same inventive concept, all however falling within the protective scope of the claims set forth hereinafter.

1. A device for electrosurgery apt to be applied onto a patient's body, comprising:
one supporting body;
four electrodes, apt to be connected in pairs to current generating means for generating an electrosurgical current, which electrodes are constrained to said supporting body and arranged at the vertexes of a quadrilateral; and
means for adjusting the relative distances of said electrodes, apt to guide translation of said electrodes in such a way that their relative distances remain in a predetermined ratio.

2. (canceled)

3. The device according to claim 1, wherein said electrodes are equidistant the one from the other and wherein said means for adjusting the relative distance of said electrodes are apt to guide the latter in such a way that they remain equidistant.

4. The device according to claim 1, wherein said supporting body is made of two parts, comprising a first and a second body overlapped and movable the one with respect to the other, and wherein said means for adjusting comprises a shape coupling between each of said electrodes and said first and second body, the arrangement being such that said shape coupling guides translation of said electrodes in such a way that, when said first and second body are moved the one with respect to the other, the electrodes are guided to move in a nearing/moving away direction, said direction depending on the sense of the relative motion between said bodies.

5. The device according to claim 4, wherein said first and second body are rotatable the one with respect to the other.

6. The device according to claim 5, wherein said shape coupling provides, for each electrode, a pair of grooves obtained onto the first and second body, respectively, the arrangement of the grooves of each pair being such that those are overlapped for an extension such as to allow reception of the respective electrode therethrough.

7. The device according to claim 6, wherein said pair of grooves consists of comprises a first groove having a substantially rectilinear development and of a second groove having a substantially arcuate development.

8. The device according to claim 4, comprising stopping means for limiting the excursion of the relative motion between said first and second body.

9. The device according to claim 4, wherein said first and second body are made of an acrylic material.

10. The device according to claim 1, wherein said supporting body has at least one graduation apt to indicate to a user the relative distance between said electrodes.

11. The device according to claim 1, wherein each of said electrodes is made of a first and of a second part electrically insulatable the one from the other.

12. The device according to claim 1, wherein said electrodes have an anti-stick coating.

13. The device according to claim 1, wherein at least one electrode of said electrodes comprises a portion having a convoluted development in order to increase the heat loss surface.

14. The device according to claim 13, wherein said portion has a substantially helicoidal development.

15. The device according to claim 1, wherein at least one electrode of said electrodes comprises an outer capsule containing a refrigeration substance, said capsule being arranged about a portion of the electrode itself.

16. An apparatus for electrosurgery of a type apt to the treatment of neoplastic tissues by thermoablation, comprising:
at least two pairs of electrodes, each electrode being apt to be arranged at a respective vertex of a quadrilateral;
current generating means comprising two generators of electrosurgical current electrically insulated the one from the other, each apt to make a thermoablation current flow between the electrodes of a respective pair arranged at opposite vertexes of said quadrilateral, so as to have a bipolar thermoablation mode wherein the thermoablation heats of the two pairs of electrodes add up at the tumour tissue to be thermoablated located in the central region of said quadrilateral; and
switching means apt to change the generator to which at least two electrodes of said pairs are connected in such a way that each of said two generators can make a thermoablation current flow between electrodes placed at adjacent vertexes of said quadrilateral.

17. (canceled)

18. (canceled)

19. The apparatus according to claim 16, wherein said generators have frequencies substantially different the one from the other.

20. The apparatus according to claim 19, wherein said frequencies differ of at least 100 kHz.

21. (canceled)

22. (canceled)

23. The apparatus according to claim 16, comprising a comb-shaped electrosurgical device, wherein each electrode of said pairs is implemented by a respective tooth of said comb-shaped device.

24. The apparatus according to claim 16, wherein each electrode of at least one pair of said pairs of electrodes is made of a first and of a second part electrically insulatable the one from the other, and wherein the apparatus comprises impedance determining means apt to enable a current measurement in a circuit comprising said first electrode parts and said current generating means and a voltage measurement, independent from said current measurement, across said second electrode parts.

25. The apparatus according to claim 16, wherein said electrodes have an anti-stick coating.

26. The apparatus according to claim 16, wherein at least one electrode of said pairs of electrodes comprises a portion having a convoluted development, in order to increase the heat dissipation surface.

27. The apparatus according to claim 26, wherein said portion has a substantially helicoidal development.

28. The apparatus according to claim 16, wherein at least one electrode of said pairs of electrodes comprises an outer capsule containing a refrigeration substance, said capsule being arranged about a portion of the electrode itself.

29. A method for the treatment of neoplastic tissues by thermoablation, comprising the steps of:
providing at least two pairs of electrodes;
providing current generating means for generating an electrosurgical current; and
making an electrosurgical current flow between each pair of electrodes according to a bipolar thermoablation mode, so as to have the thermoablation heats of the two pairs of electrodes add up at the tumour tissue to be thermoablated.

30. The method according to claim 29, wherein said thermoablation currents of the two pairs of electrodes have substantially different frequencies.

31. The method according to the claim 30, wherein said frequencies differ of at least 100 kHz.

32. The method according to claim 29, wherein the electrodes of said pairs are placed onto a patient's body at the vertexes of a quadrilateral, the electrodes of each pair being placed at opposed vertexes of said quadrilateral.

33. The method according to claim 29, wherein said step (c) provides the devitalization of the tumour mass.

34. The method according to claim 29, wherein said step (c) provides the devitalization of a layer of tissue surrounding the tumour mass.

35. The method according to claim 29, wherein the at least two pairs of electrodes are adapted to be arranged at the respective vertices of a quadrilateral and where the current generating means comprise two generators of electrosurgical current electrically insulated the one from the other, each apt to make a thermoablation current flow between the electrodes of a respective pair arranged at opposite vertexes of said quadrilateral, so as to have a bipolar thermoablation mode wherein the thermoablation heats of the two pairs of electrodes add up at the tumour tissue to be thermoablated located in the central region of said quadrilateral, and a switching means apt to change the generator to which at least two electrodes of said pairs are connected in such a way that each of said two generators can make a thermoablation current flow between electrodes placed at adjacent vertices of said quadrilateral.

36. An apparatus for the treatment of neoplastic tissues by thermoablation, comprising:
at least two pairs of electrodes coupled to a current generator adapted to make a thermoablation current flow between the electrodes of a respective pair when the electrodes are emplaced generally around a selected neoplastic tissue, the opposed electrodes of each respective pair being placed about the neoplastic tissue such that the respective currents flowing therebetween constructively interfere the one with the other.

37. The apparatus according to claim 36, wherein the current generator comprises at least two distinct current generators, the one being electrically insulated from the other, each of the current generators being adapted induce a current to flow between the electrodes of a respective pair arranged at opposite vertexes of a quadrilateral.

38. The apparatus according to claim 36, further comprising:
a switch coupled between the electrodes and the current generator that is adapted to selectively couple the electrodes to the current generator in distinct pairs.

39. The apparatus according to claim 38, wherein the at least two pairs of electrodes are arranged at the vertices of a regular polygon having a number of vertices equal to the number of electrodes.

40. The apparatus according to claim 39, wherein the apparatus has four electrodes and the regular polygon is a square.

41. The apparatus according to claim 38, wherein the at least two pairs of electrodes are arranged around a perimeter of the neoplastic tissue.

42. The apparatus according to claim 39, wherein the apparatus has four electrodes and the regular polygon is generally rectangular.

43. The apparatus according to claim 37, wherein said at least two distinct current generators have frequencies substantially different the one from the other.

44. The apparatus according to claim 43, wherein said frequencies differ of at least 100 kHz.

45. The apparatus according to claim 36, comprising a comb-shaped electrosurgical device having a plurality of teeth, wherein each electrode of said pairs is implemented by a respective tooth of said comb-shaped device.

46. The apparatus according to claim 36, wherein each electrode of at least one pair of said pairs of electrodes is made of a first and of a second part electrically insulated the one from the other, and wherein the apparatus comprises impedance determining apparatus adapted to enable a current measurement in a circuit comprising said first electrode parts and said current generator and a voltage measurement, independent from said current measurement, across said second electrode parts.

47. The apparatus according to claim 36, wherein said electrodes have an anti-stick coating.

48. The apparatus according to claim 36, wherein at least one electrode of said pairs of electrodes comprises a portion having a convoluted development.

49. The apparatus according to claim 48, wherein said portion has a substantially helicoidal development.

50. The apparatus according to claim 36, wherein at least one electrode of said pairs of electrodes comprises an outer capsule containing a refrigeration substance, said capsule being arranged about a portion of the electrode itself.
